(19) United States
(12) Patent Application Publication
(10) Pub. No.: US 2010/0184873 A1
(43) Pub. Date: Jul. 22, 2010

(54) MULTI STAGE PROCESS FOR PRODUCING HYDROCARBONS FROM SYNGAS

(76) Inventors: Maarten Bracht, Amsterdam (NL); Maarten Hesselink, Amsterdam (NL); Christiaan Nijst, Amsterdam (NL); Thomas Joris Remans, Amsterdam (NL)

Correspondence Address: SHELL OIL COMPANY, P O BOX 2463, HOUSTON, TX, 772522463, US

(21) Appl. No.: 12/640,298

(22) Filed: Dec. 17, 2009

(30) Foreign Application Priority Data
Dec. 18, 2008 (EP) 08172170.6

(51) Int. Cl. C07C 27/06

(52) U.S. Cl. 518/706

(57) ABSTRACT

The invention pertains to a multi-stage process for the production of hydrocarbons from syngas comprising hydrogen and carbon monoxide, which comprises the steps of: a) providing a fresh syngas feed to a first stage Fischer-Tropsch reactor and allowing CO and hydrogen to convert into hydrocarbon products at a temperature in the range from 125 to 400° C. and a pressure in the range from 5 to 150 bar absolute, and a gaseous hourly space velocity in the range from 500 to 10000 Nl/l/h; b) feeding the effluent from the first stage reactor to a separation unit; c) removing a gasous effluent stream comprising hydrogen and CO from the separation unit; d) removing one or more other streams comprising hydrocarbon and/or water from the separation unit; e) conveying a first portion of the gaseous effluent stream to a second stage Fischer-Tropsch reactor and allowing CO and hydrogen to convert into hydrocarbon products in the second stage Fischer-Tropsch reactor at a temperature in the range from 125 to 400° C. and a pressure in the range from 5 to 150 bar absolute, and a gaseous hourly space velocity in the range from 500 to 10000 Nl/l/h, whereby the first stage Fischer-Tropsch reactor and the second stage Fischer-Tropsch reactor are separate reactors; f) feeding the effluent from the second stage Fischer-Tropsch reactor to the separation unit; g) removing a second portion of the gaseous effluent stream as off-gas.

This application claims the benefit of European Application No. 08172170.6 filed Dec. 18, 2008 which is incorporated herein by reference.

### BACKGROUND OF THE INVENTION

The present invention relates to a multi-stage process for the production of hydrocarbon products from syngas.

The manufacture and further processing of syngas has been found to be an attractive manner for processing various types of hydrocarbonaceous feedstock.

A first source for the manufacture of syngas are light hydrocarbon feeds, especially methane from natural sources, for example natural gas, associated gas and/or coal bed methane. There is not always the option to use the gas at its source. Transportation of gas, for example through a pipeline or in the form of liquefied natural gas, requires extremely high capital expenditure or is simply not practical. This holds true even more in the case of relatively small gas production rates and/or fields. Re-injection of gas will add to the costs of oil production, and may, in the case of associated gas, result in undesired effects on crude oil production. Burning of associated gas has become an undesirable option in view of depletion of hydrocarbon sources and air pollution. One of the ways to process this gas is the conversion into syngas.

A further source for the manufacture of syngas are the very heavy hydrocarbon fractions, or feedstock which is difficult to process by other means. Examples of this type of feedstock include peat, biomass, or coal. These materials can also be converted to syngas, for example via the Shell gasification process.

The syngas manufactured from the above, or other, sources, can be converted in one or more steps over a suitable catalyst at elevated temperature and pressure into mainly paraffinic compounds ranging from methane to high molecular weight molecules comprising up to 200 carbon atoms, or, under particular circumstances, even more.

WO2007/009952 describes a multi-stage process for the production of hydrocarbon products from syngas, each stage of the process comprising one or more syngas conversion reactors in which syngas is partially converted into hydrocarbon products at conversion conditions. Part of the syngas stream is obtained from a partial oxidation process. Another syngas stream is a recycle stream from the conversion reactor.

WO2008/062208 describes a process for converting synthesis gas to hydrocarbons, using a Fischer-Tropsch synthesis wherein two Fisher-Tropsch reactors are used in series with water removal between them and additional hydrogen added to the second reactor.

There are various problems associated with the processes described in these references. In the first place, they are difficult to adapt to be suitable for different operations with different syngas compositions, therewith necessitating different process designs for different units. A process design which can be applied at different locations would be attractive. Further, the processes described in the above references are relatively inflexible in that it is difficult within the existing operational set-up to compensate for process irregularities, such as reactors being regenerated, or otherwise off-line, and for other variations in the process.

It has now been found that this problem can be solved by the process according to the invention.

### SUMMARY OF THE INVENTION

The present invention provides a multi-stage process for the production of hydrocarbons from syngas comprising hydrogen and carbon monoxide, which comprises the steps of:
a) providing a fresh syngas feed to a first stage Fischer-Tropsch reactor and allowing CO and hydrogen to convert into hydrocarbon products at a temperature in the range from 125 to 400° C., preferably 175 to 300° C., and a pressure in the range from 5 to 150 bar absolute, and a gaseous hourly space velocity in the range from 500 to 10000 Nl/l/h;
b) feeding the effluent from the first stage reactor to a separation unit;
c) removing a gasous effluent stream comprising hydrogen and CO from the separation unit;
d) removing one or more other streams comprising hydrocarbon and/or water from the separation unit;
e) conveying a first portion of the gaseous effluent stream to a second stage Fischer-Tropsch reactor and allowing CO and hydrogen to convert into hydrocarbon products in the second stage Fischer-Tropsch reactor at a temperature in the range from 125 to 400° C., preferably 175 to 300° C., and a pressure in the range from 5 to 150 bar absolute, and a gaseous hourly space velocity in the range from 500 to 10000 Nl/l/h,
   whereby the first stage Fischer-Tropsch reactor and the second stage Fischer-Tropsch reactor are separate reactors;
f) feeding the effluent from the second stage Fischer-Tropsch reactor to the separation unit;
g) removing a second portion of the gaseous effluent stream as off-gas.

The effluent from the second stage Fischer-Tropsch reactor is thus fed, in step f), to the same separation unit as to which the effluent from the first stage reactor is fed in step b).

The Fischer-Tropsch reactors used in the present invention contain a Fischer-Tropsch catalyst. Preferably the Fischer-Tropsch catalyst comprises a Group VIII metal component, more preferably cobalt, iron and/or ruthenium, most preferably cobalt. References to the Periodic Table and groups thereof used herein refer to the previous IUPAC version of the Periodic Table of Elements such as that described in the 68th Edition of the Handbook of Chemistry and Physics (CPC Press).

Typically, the catalysts comprise a catalyst carrier. The catalyst carrier is preferably porous, such as a porous inorganic refractory oxide, more preferably alumina, silica, titania, zirconia or combinations thereof, most preferably titania.

The optimum amount of catalytically active metal present on the carrier depends inter alia on the specific catalytically active metal. Typically, the amount of cobalt present in the catalyst may range from 1 to 100 parts by weight per 100 parts by weight of carrier material, preferably from 10 to 50 parts by weight per 100 parts by weight of carrier material. In case the catalyst comprises cobalt and titania, the amount of cobalt preferably is in the range of between 10 weight percent (wt %) and 35 wt % cobalt, more preferably between 15 wt % and 30 wt % cobalt, calculated on the total weight of titania and cobalt.

### BRIEF DESCRIPTION OF THE DRAWINGS

A number of embodiments of the invention are described in detail and by way of example only with reference to the accompanying drawings.

FIG. 1 provides a first embodiment of the process according to the invention.

FIG. 2 provides a second embodiment of the process according to the invention.

### DETAILED DESCRIPTION OF THE INVENTION

One of the features of the present invention is that the effluent of the first stage Fischer-Tropsch reactor and that of the second stage Fischer-Tropsch reactor are fed to the same separation unit, and that portions of the recycle stream from said separation unit are fed to both the first stage and the second stage Fischer-Tropsch reactor. This feature results in a number of advantages being attained.

In the first place, it has been found that the process of the present invention results in a high conversion, accompanied by a high selectivity to C5+ products. Further, the process of the present invention can be adapted to various syngas compositions, which allows the use of the same design in different operations. Further, as will be discussed in more detail below, the use of a single separation unit for both the first stage and the second stage operation makes it easier to compensate for reactors being off-line, etc. Additionally, the combination of the separation units and the recycle streams makes the process less complicated than a system where two separation units are provided, and therewith more economical to build and to operate.

Another advantage of the process of the present invention is that it allows the use of syngas with a relatively low hydrogen/CO ratio, which material cannot be processed by all conventional Fischer-Tropsch operations. The syngas composition may, for example, be controlled by choosing a certain type of feedstock in the syngas manufacturing. In general, when syngas is prepared from biomass or coal, a lower hydrogen/CO ratio can be obtained as compared to syngas prepared from natural gas.

Further advantages of the present invention and specific embodiments thereof will become apparent from the further specification.

In the context of the present specification a first-stage Fischer-Tropsch reactor is a reactor which is provided with fresh syngas feed, which generally has a hydrogen/CO ratio in the range of 0.3 to 2.3:1, more in particular in the range of 0.5 to 2.1:1. Optionally, the first-stage Fischer-Tropsch reactor is provided with a recycle stream of gaseous effluent from the separation unit.

In the context of the present specification a second-stage Fischer-Tropsch reactor is a reactor which is provided with a recycle stream of gaseous effluent from the separation unit. Optionally, the second-stage Fischer-Tropsch reactor is provided with an additional hydrogen-containing gas stream. The hydrogen/CO ratio of the hydrogen-containing gas stream generally is between 2:1 and infinite, more in particular between 3:1 and infinite.

The hydrogen/CO ratio of the syngas fed to the first-stage reactor is lower than the hydrogen/CO ratio of the hydrogen-containing gas stream that may be fed to the second-stage reactor.

If so desired, the process may be adapted to also include third- and further-stage reactors, the effluent of which may or may not be fed to the same separation unit. In case a third-, or a third- and one or more further-stage reactors, is/are included, the first stage, and the second stage, the third stage, and any further stage

Fischer-Tropsch reactor are separate reactors. For reasons of process efficiency it is preferred for the effluent of any third- and further-stage reactors to be fed to the same separation unit as the effluent of the first- and second-stage reactors. The third- and further-stage reactors may or may not be fed with an additional hydrogen-containing gas stream.

The invention will be discussed in more detail below.

The first step of the process according to the invention is providing a syngas feed to a first stage Fischer-Tropsch reactor in which syngas is partially converted into hydrocarbon products under conversion conditions. The temperature is in the range from 125 to 400° C. and a pressure in the range from 5 to 150 bar absolute, and a gaseous hourly space velocity in the range from 500 to 10000 Nl/l/h.

The starting material in the process according to the invention is fresh syngas, comprising hydrogen and CO. The syngas may, for example, be obtained from natural gas, but also from peat, coal, biomass, or other hydrocarbon fractions by processes like gasification, autotherm reforming, catalytic or non-catalytic partial oxidation.

The syngas as fed to the first stage Fischer-Tropsch reactor generally has a hydrogen/CO ratio in the range of 0.3 to 2.3:1, more in particular in the range of 0.5 to 2.1:1.

In one embodiment, syngas is used which has a relatively high hydrogen/CO ratio, for example in the range of 1.0 to 2.3:1, in particular in the range of 1.5 to 2.1:1, still more in particular in the range of 1.7 to 2.0:1. Syngas with a relatively high hydrogen/CO ratio may, for example be obtained from light hydrocarbon feeds, for example natural gas sources.

In another embodiment, syngas is used which has a relatively low hydrogen/CO ratio, for example in the range of 0.3 to 1.6:1, in particular in the range of 0.5 to 1.6:1, still more in particular in the range of 0.9-1.6:1. Syngas with a relatively low hydrogen/CO ratio may, for example be obtained from heavy hydrocarbonaceous sources like peat, coal, and biomass. The fact that the present invention allows the use of syngas with a relatively low hydrogen/CO ratio is a particular advantage of the present invention, as this material cannot be processed by all conventional Fischer-Tropsch operations.

Numerous types of reactor systems have been developed for carrying out the Fischer-Tropsch reaction. For example, Fischer-Tropsch reactor systems include fixed bed reactors, especially multitubular fixed bed reactors, fluidized bed reactors, such as entrained fluidized bed reactors and fixed fluidized bed reactors, and slurry bed reactors such as three-phase slurry bubble columns and ebullated bed reactors. The present invention is applicable to all types of reactor systems. The use of multitubular reactors and slurry bed reactors may be mentioned in particular.

In the reactor, the syngas is partially converted into hydrocarbon products under conversion conditions, accompanied by the formation of water. This is generally done by contacting the syngas with a catalyst. Suitable catalysts are known in the art and will be discussed below. Suitably the conversion of CO that is fed to the reactor (in the combined feed of fresh feed and optionally recycle gas) is between 20 and 90%, preferably 25-70%, more preferably 30-50%. The CO conversion can easily be changed by increasing or decreasing the reactor temperature and/or the reaction pressure, and by adapting the recycle conditions.

The effluent from the first stage reactor is fed to a separation unit, where it is separated to form a gaseous effluent stream and one or more other streams which comprise hydrocarbon and/or water.

The gaseous effluent stream comprises hydrogen and CO. The hydrogen/CO ratio of the gaseous effluent stream generally is between 0 and 1.5:1, more in particular between 0.1 and 0.9:1, still more in particular between 0.3 and 0.9:1. The gaseous effluent stream may also comprise other components such as inert gases like nitrogen. Carbon dioxide and amounts of C1-C4 hydrocarbons may also be present.

The stream or streams comprising hydrocarbon comprise(s) a C5+ hydrocarbon product, with optionally lower hydrocarbon fractions dissolved therein.

The separation unit may be a single separation unit or more than one separation units connected together. Suitable separation units include gas/liquid separators. It is within the scope of the skilled person to select an appropriate separation system and separation conditions. Depending on the separator configuration, the water and the hydrocarbons may be present in a combined stream or in different streams.

The stream(s) which comprise(s) hydrocarbon and/or water is/are removed from the separation unit, as is the gaseous effluent stream comprising hydrogen and CO.

The gaseous effluent stream comprising hydrogen and CO is divided into a number of portions, for example by way of a splitter.

In the present invention, a first portion of the gaseous effluent stream from the separation unit is fed to the second stage Fisher-Tropsch reactor, and a second portion of the gaseous effluent stream from the separation unit is removed as off-gas.

In one embodiment of the present invention, a third portion of the gaseous effluent stream is conveyed to the first stage Fischer-Tropsch reactor. Whether or not, and in how far, this recycle stream will be applied will depend on the hydrogen/CO ratio of the fresh feed. One of the features of the recycle is that it lowers the hydrogen/CO ratio in the actual feed to the reactor.

It is a feature of the present invention that the recycle stream can be divided at will to form the recycle stream to the second stage Fischer-Tropsch reactor, the off-gas stream, and, if so desired, the recycle stream to the first stage Fischer-Tropsch reactor. This allows detailed tailoring of the composition of the recycle streams, which can be used to compensate for process variations due to, for example, reactors being off-line, and, in operation design, to address the composition of the syngas at a particular location.

A first portion of the gaseous effluent is conveyed to a second stage Fischer-Tropsch reactor in which CO and hydrogen are partially converted into hydrocarbon products under conversion conditions. The temperature is in the range from 125 to 400° C., preferably 175 to 300° C., and a pressure in the range from 5 to 150 bar absolute, and a gaseous hourly space velocity in the range from 500 to 10000 Nl/l/h. For properties of the reactor, reference is made to what has been stated above for the first stage reactor.

If so desired, a hydrogen-containing gas stream is provided to the second stage Fischer-Tropsch reactor. This may, for example, be a syngas stream or another stream which contains hydrogen, depending on the content of the recycle stream. The hydrogen/CO ratio of the hydrogen-containing gas stream, if present, generally is between 2:1 and infinite, more in particular between 2 and 200:1, still more in particular between 3 and 100:1. If a hydrogen-containing gas stream is provided to the second stage reactor, it may be used to adjust the conversion of the second stage reactor. For such an adjustment, the composition and feed rate of the hydrogen-containing gas stream may be attuned.

In the second stage reactor, the carbon monoxide, which originates from the recycle stream and optionally from the hydrogen-containing stream, is partially converted into hydrocarbon products under conversion conditions. This is generally done with a catalyst. Suitably the conversion of CO that is fed to the reactor (in the combined feed of recycle gas and optionally additional hydrogen-containing gas) is between 20 and 90%, preferably 25-70%, more preferably 30-50%.

The CO conversion can easily be changed by increasing or decreasing the reactor temperature and/or the reaction pressure, and by adapting the recycle conditions.

The effluent from the second stage Fischer-Tropsch reactor is fed to the separation unit.

As indicated above, gaseous effluent from the separation unit may or may not be recycled to the first stage In the case that gaseous effluent from the separation unit is recycled to the first stage, the ratio between fresh syngas and recycle gaseous effluent is suitably between 0.1 and 10:1, preferably between 0.2 and 5:1, more preferably between 0.3 and 3:1.

The hydrogen/CO ratio of the combined feed to the first stage reactor (comprising fresh syngas and optionally recycle gaseous effluent) is generally in the range of 0.1-2.3:1, more in particular in the range of 0.5-2.1:1.

As indicated above, additional hydrogen-containing gas may or may not be provided to the second stage reactor. In the case that additional hydrogen-containing gas is provided, the ratio between the additional hydrogen-containing gas and the recycle gaseous effluent originating from the separation unit generally is between 0.1 and 10:1, more in particular between 0.2 and 5:1.

The hydrogen/CO ratio of the combined feed to the second stage reactor (comprising recycle gaseous effluent and optionally additional hydrogen-containing gas) is generally in the range of 0.1-2.3:1, more in particular in the range of 0.5-2.1:1. The hydrogen/CO ratio of the combined feed to the second-stage reactor may be the same or different as the hydrogen/CO ratio of the combined feed to the first-stage reactor.

In general, the upper limit of the hydrogen/CO ratio will be the usage ratio of the unit. The usage ratio of the unit is the ratio in which hydrogen and CO are used in a reactor. It depends, in al., on the nature of the catalyst and the process conditions applied.

It is noted that in the present specification ratio's between gas streams are volume ratio's, and hydrogen/CO ratios are molar ratios.

In one embodiment of the present invention, the first and/or the second portions of the gaseous effluent stream are subjected to a water removal step before being fed to the first and/or second stage Fischer-Tropsch reactors. This is because the presence of water may detrimentally affect the conversion conditions in the Fisher-Tropsch reactor and may reduce process efficiency. Whether a water removal step is required will also depend on the efficiency of the water removal in the separation unit. Generally, the water content of the recycle streams to the first and second stage reactor is at most 10 vol. %, in particular at most 5 vol. %. Suitable water removal apparatus is known in the art and requires no further elucidation.

In one embodiment of the present invention, the first and/or the second stage Fischer-Tropsch reactor may comprise a set of two or more subreactors operated in parallel. Therefore, where in the present specification reference is made to, for example, the first stage Fischer-Tropsch reactor, this also encompasses a set of two or more first stage Fischer-Tropsch reactors operated in parallel.

Where the first stage Fischer-Tropsch reactor comprises a set of two or more subreactors operated in parallel, the set preferably contains at least 2, more preferably at least 4 reactors. The maximum number of reactors is generally not critical to the present invention. It is determined by the required production volume and reactor production capacity.

The number of subreactors in the second stage Fischer-Tropsch reactor may vary between 10 and 200% of the number of Fischer-Tropsch reactors in the first stage.

One embodiment of the present invention provides a process with a flexibility which is even more improved. In this embodiment, a first-stage reactor is used which comprises at least two subreactors and a second-stage reactor is used which comprises at least two subreactors, wherein fresh syngas feed lines, additional hydrogen-containing gas feed lines, and recycle gaseous effluent feed lines to both the first stage subreactors and the second stage subreactors. In operation, each reactor will be provided with either fresh syngas or additional hydrogen-containing gas. This in essence allows the selection of which reactor will operate as first stage reactor (that is, will be provided with fresh syngas feed and optionally gaseous effluent from the separation unit), and which reactor will operate as second stage reactor (that is, will be provided with gaseous effluent from the separation unit and additional hydrogen-containing gas).

Accordingly, the present invention also pertains to a multi-stage process for the production of hydrocarbons from syngas comprising hydrogen and carbon monoxide, which comprises the steps of:
a) providing at least three Fischer-Tropsch subreactors, wherein each subreactor is connected on the inlet side to a feed for fresh syngas feed, a feed for an additional hydrogen-containing gas, and a feed for recycle gas and wherein each subreactor is connected on the outlet side to an effluent line, wherein all effluent lines are connected to a separation unit;
b) providing a fresh syngas feed to at least one and less than all Fischer-Tropsch subreactors, and allowing CO and hydrogen to convert in these subreactor(s) into hydrocarbon products in the second stage Fischer-Tropsch reactor at a temperature in the range from 125 to 400° C., preferably 175 to 300° C., and a pressure in the range from 5 to 150 bar absolute, and a gaseous hourly space velocity in the range from 500 to 10000 Nl/l/h,
   wherein the subreactors provided with fresh syngas feed are not simultaneously provided with additional hydrogen-containing gas;
c) feeding the effluent from the subreactors provided with fresh syngas to a separation unit;
d) removing a gasous effluent stream comprising hydrogen and CO from the separation unit;
e) removing one or more other streams comprising hydrocarbon and/or water from the separation unit;
f) conveying a first portion of the gaseous effluent stream to the at least one and less than all Fischer-Tropsch subreactors which are not provided with fresh syngas, and allowing CO and hydrogen to convert in these subreactor(s) into hydrocarbon products in the second stage Fischer-Tropsch reactor at a temperature in the range from 125 to 400° C., preferably 175 to 300° C., and a pressure in the range from 5 to 150 bar absolute, and a gaseous hourly space velocity in the range from 500 to 10000 Nl/l/h;
g) feeding the effluent from the subreactors provided with a first portion of the gaseous effluent stream to the separation unit;
h) removing a second portion of the gaseous effluent stream as off-gas.

The effluent from the subreactors provided with a first portion of the gaseous effluent stream is thus fed, in step g), to the same separation unit as to which the effluent from the subreactors provided with fresh syngas is fed in step c).

The Fischer-Tropsch reactors used in the present invention contain a Fischer-Tropsch catalyst. Preferably the Fischer-Tropsch catalyst comprises a Group VIII metal component, more preferably cobalt, iron and/or ruthenium, most preferably cobalt.

Typically, the catalysts comprise a catalyst carrier. The catalyst carrier is preferably porous, such as a porous inorganic refractory oxide, more preferably alumina, silica, titania, zirconia or combinations thereof, most preferably titania.

The optimum amount of catalytically active metal present on the carrier depends inter alia on the specific catalytically active metal. Typically, the amount of cobalt present in the catalyst may range from 1 to 100 parts by weight per 100 parts by weight of carrier material, preferably from 10 to 50 parts by weight per 100 parts by weight of carrier material. In case the catalyst comprises cobalt and titania, the amount of cobalt preferably is in the range of between 10 weight percent (wt %) and 35 wt % cobalt, more preferably between 15 wt % and 30 wt % cobalt, calculated on the total weight of titania and cobalt.

In one embodiment, a third portion of the gaseous effluent stream is conveyed to the at least one and less than all Fischer-Tropsch subreactors which are being provided with fresh syngas feed.

In one embodiment, an additional hydrogen-containing gas stream is provided to the at least one and less than all Fischer-Tropsch subreactors which are not provided with fresh syngas.

As is known to the skilled person, fresh Fischer-Tropsch catalyst is relatively sensitive. Therefore, it is often desired to initially operate a Fischer-Tropsch catalyst under certain operating conditions, and after a certain period of time use the catalyst under operating conditions which are more severe than the initial operating conditions.

In a preferred embodiment of the process according to the present invention, it is possible to have less severe operating conditions at second-stage then at first-stage. In such an embodiment of the process it may be desirable to first operate a Fischer-Tropsch catalyst under second-stage operating conditions which are less severe than first-stage operating conditions, and after a certain period of time use the catalyst under first-stage operation conditions.

The embodiment of the present invention, where all subreactors are connected to the same separator, and all subreactors are connected to the syngas feed line, the line with recycle gaseous effluent from the separation unit, and the line with the additional hydrogen-containing gas, allows shifting individual reactors from second stage operation to first-stage operation by the mere switching of valves. A further advantage of this embodiment is that in the case of changing process conditions, e.g., in the case of a reactor being off-line, it is relatively easy to compensate therefor by adapting the feed streams to the respective units. Thus, in one embodiment of the present invention, during operation for at least one of the subreactors which are provided with a first portion of the gaseous effluent stream and an additional hydrogen-containing gas stream, the provision of an additional hydrogen-containing gas stream is discontinued, and the provision of a fresh syngas feed is started. This is in effect the switching of the subreactor from second stage operation to first stage operation.

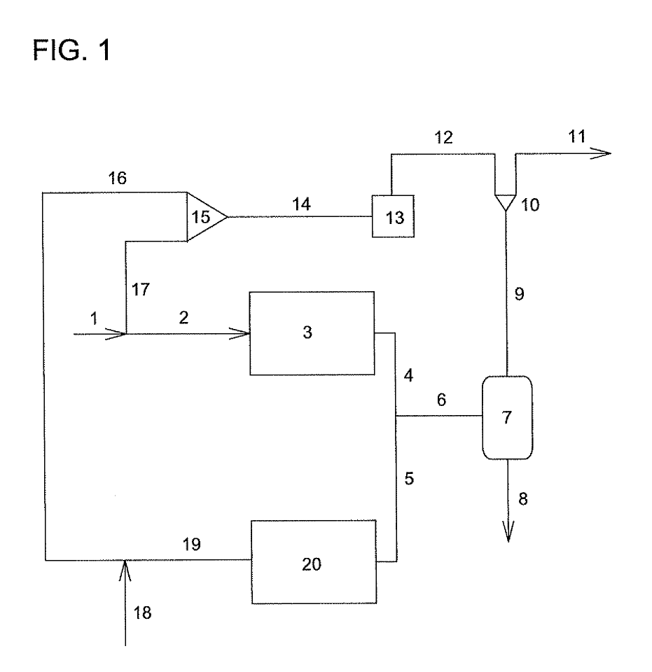

One embodiment of the process according to the invention is illustrated in FIG. 1. Fresh syngas, comprising CO and hydrogen, provided through feed line (1), is combined with recycle gas provided through line (17) to form a combined feed (2), which is led to a first stage Fischer-Tropsch reactor (3). The effluent from the first stage Fischer-Tropsch reactor (3) is withdrawn through line (4), and combined with effluent stream (5) from the second stage Fischer-Tropsch reactor (20) to form a combined effluent (6), which is led to separation unit (7).

In separation unit (7), the Fischer-Tropsch effluent is separated to form Fischer-Tropsch liquid products, which are withdrawn through line (8), and a gaseous effluent stream which is fed through line (9) to a splitter (10). In splitter (10), an off-gas stream (11) is withdrawn, and a recycle stream (12) is led to a compressor (13). The resulting stream (14) is led to a splitter (15) where it is split in a first portion of gaseous effluent (16) which is provided to the second stage Fischer-Tropsch reactor (20) and a second portion of gaseous effluent (17), which is recycled to the first stage Fischer-Tropsch reactor (3). The first portion of gaseous effluent (16) is combined with an additional hydrogen-containing gas stream through line (18), and the combined stream (19) is fed to the second stage Fischer-Tropsch reactor (20).

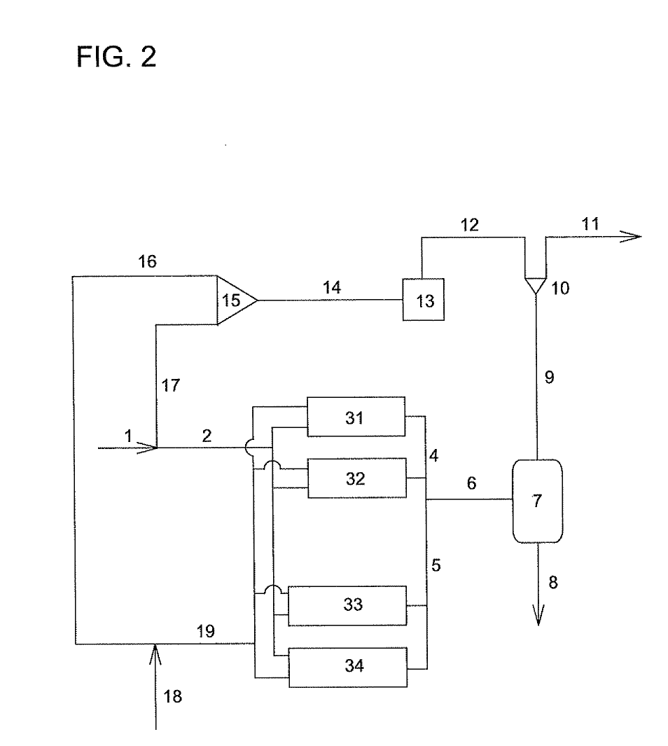

A further embodiment of the present invention is illustrated in FIG. 2. Fresh syngas, comprising CO and hydrogen, provided through feed line (1), is combined with recycle gas provided through line (17) to form a combined feed (2). The line for combined feed (2) is connected to subreactors (31), (32) (33) and (34). The subreactors are provided with effluent withdrawal lines (4), (5), which are combined to form a combined effluent (6), which is led to separation unit (7).

In separation unit (7), the Fischer-Tropsch effluent is separated to form Fischer-Tropsch liquid products, which are withdrawn through line (8), and a gaseous effluent stream which is fed through line (9) to a splitter (10). In splitter (10), an off-gas stream (11) is withdrawn, and a recycle stream (12) is led to a compressor (13). The resulting stream (14) is led to a splitter (15) where it is split into a first portion of gaseous effluent (16) which is connected to a feed for the provisional of an additional hydrogen-containing gas through line (18), to form a combined feed line (19), which is connected with all subreactors (31), (32), (33), and (34). A second portion is provided through line (17), and combined with fresh syngas feed (1), as discussed above.

In operation, at least one but less than all subreactors (31), (32), (33), and (34) are provided with combined feed (2) with feed (19) being shut off. These subreactors operate in first-stage mode. The other at least one but less than all subreactors (31), (32), (33), and (34) are provided with feed (19) with combined feed (2) being shut off. These subreactors operate in second-stage mode.

In the embodiment presented in FIG. 2, syngas feed (1) and recycle gas (17) are mixed to form combined feed (2) which is then split to the various reactors. In an alternative embodiment, the syngas feed stream and recycle gas stream are first split into individual streams, and then combined to form combined feed streams to the individual reactors.

The Fischer-Tropsch synthesis is preferably carried out at a temperature in the range from 125 to 400° C., more preferably 175 to 300° C., most preferably 200 to 260° C. The pressure preferably ranges from 5 to 150 bar absolute, more preferably from 20 to 80 bar absolute. The gaseous hourly space velocity may vary within wide ranges and is typically in the range from 500 to 10000 Nl/l/h, preferably in the range from 1500 to 4000 Nl/l/h.

The conditions and/or parameters for first and second stage reactors may be the same or different. Such differences include reactor temperatures and pressures used, the H₂/CO entry and exit ratios. Also as the nature of the catalyst may be the same or different for first and second stage reactors.

Products of the Fischer-Tropsch synthesis may range from methane to heavy hydrocarbons. Preferably, the production of methane is minimized and a substantial portion of the hydrocarbons produced have a carbon chain length of a least 5 carbon atoms. It has been found that in the process of the invention a high selectivity can be obtained in combination with a high overall conversion. More in particular, the selectivity may be such that the amount of C5+ hydrocarbons is at least 60% by weight of the total product, more preferably, at least 70% by weight, even more preferably, at least 80% by weight, most preferably at least 85% by weight. The process according to the invention can give an overall conversion of at least 80%, more in particular of at least 85%, sometimes even at least 90%, calculated from the CO of the fresh syngas.

The products obtained via the process according to the invention can be processed through hydrocarbon conversion and separation processes known in the art to obtain specific hydrocarbon fractions. Suitable processes are for instance hydrocracking, hydroisomerisation, hydrogenation and catalytic dewaxing. Specific hydrocarbon fractions are for instance LPG, naphtha, detergent feedstock, solvents, drilling fluids, kerosene, gasoil, base oil and waxes.

The various separation and water removal steps can be carried out using procedures known in the art, which require no further elucidation here.

The Fischer-Tropsch reactors used in the present invention contain a Fischer-Tropsch catalyst. Fisher-Tropsch catalysts are known in the art. They typically comprise a Group VIII metal component, preferably cobalt, iron and/or ruthenium, more preferably cobalt. Typically, the catalysts comprise a catalyst carrier. The catalyst carrier is preferably porous, such as a porous inorganic refractory oxide, more preferably alumina, silica, titania, zirconia or combinations thereof. References to the Periodic Table and groups thereof used herein refer to the previous IUPAC version of the Periodic Table of Elements such as that described in the 68th Edition of the Handbook of Chemistry and Physics (CPC Press).

The optimum amount of catalytically active metal present on the carrier depends inter alia on the specific catalytically active metal. Typically, the amount of cobalt present in the catalyst may range from 1 to 100 parts by weight per 100 parts by weight of carrier material, preferably from 10 to 50 parts by weight per 100 parts by weight of carrier material.

The catalytically active metal may be present in the catalyst together with one or more metal promoters or co- catalysts. The promoters may be present as metals or as the metal oxide, depending upon the particular promoter concerned. Suitable promoters include oxides of metals from Groups IA, IB, IVB, VB, VIB and/or VIIB of the Periodic Table, oxides of the lanthanides and/or the actinides. Preferably, the catalyst comprises at least one of an element in Group IVB, VB and/or VIIB of the Periodic Table, in particular titanium, zirconium, maganese and/or vanadium. As an alternative or in addition to the metal oxide promoter, the catalyst may comprise a metal promoter selected from Groups VIIB and/or VIII of the Periodic Table. Preferred metal promoters include rhenium, platinum and palladium.

A most suitable catalyst comprises cobalt as the catalytically active metal and zirconium as a promoter. Another most suitable catalyst comprises cobalt as the catalytically active metal and manganese and/or vanadium as a promoter. The promoter, if present in the catalyst, is typically present in an amount of from 0.1 to 60 parts by weight per 100 parts by weight of carrier material. It will however be appreciated that the optimum amount of promoter may vary for the respective elements which act as promoter. If the catalyst comprises cobalt as the catalytically active metal and manganese and/or vanadium as promoter, the cobalt:(manganese +vanadium) atomic ratio is advantageously at least 12:1.

It will be understood that it is within the scope of the skilled person to determine and select the most appropriate conditions for a specific reactor configuration and reaction regime.

What is claimed is:

1. A multi-stage process for the production of hydrocarbons from syngas comprising hydrogen and carbon monoxide, which comprises the steps of:
a) providing a fresh syngas feed to a first stage Fischer-Tropsch reactor and allowing CO and hydrogen to convert into hydrocarbon products at a temperature in the range from 125 to 400° C. and a pressure in the range from 5 to 150 bar absolute, and a gaseous hourly space velocity in the range from 500 to 10000 Nl/l/h;
b) feeding the effluent from the first stage reactor to a separation unit;
c) removing a gasous effluent stream comprising hydrogen and CO from the separation unit;
d) removing one or more other streams comprising hydrocarbon and/or water from the separation unit;
e) conveying a first portion of the gaseous effluent stream to a second stage Fischer-Tropsch reactor and allowing CO and hydrogen to convert into hydrocarbon products in the second stage Fischer-Tropsch reactor at a temperature in the range from 125 to 400° C. and a pressure in the range from 5 to 150 bar absolute, and a gaseous hourly space velocity in the range from 500 to 10000 Nl/l/h, whereby the first stage Fischer-Tropsch reactor and the second stage Fischer-Tropsch reactor are separate reactors;
f) feeding the effluent from the second stage Fischer-Tropsch reactor to the separation unit; and
g) removing a second portion of the gaseous effluent stream as off-gas.

2. A process according to claim 1, wherein a third portion of the gaseous effluent stream is conveyed to the first stage Fischer-Tropsch reactor.

3. A process according to claim 1, wherein an additional hydrogen-containing gas stream is provided to the second stage Fischer-Tropsch reactor.

4. A process according to claim 3, wherein the hydrogen-containing gas stream which is provided to the second stage Fischer-Tropsch reactor is a syngas stream comprising hydrogen and carbon monoxide.

5. A process according to claim 1, wherein the first stage Fischer-Tropsch reactor comprises a set of two or more subreactors operated in parallel.

6. A process according to claim 1, wherein the second stage Fischer-Tropsch reactor comprises a set of two or more subreactors operated in parallel.

7. A process according to claim 1, wherein the syngas as fed to the first stage Fischer-Tropsch reactor has a hydrogen/CO ratio in the range of 0.3 to 2.3:1.

8. A process according to claim 6, wherein the syngas as fed to the first stage Fischer-Tropsch reactor has a hydrogen/CO ratio in the range of 1.0 to 2.3:1.

9. A process according to claim 1, wherein the syngas as fed to the first stage Fischer-Tropsch reactor has a hydrogen/CO ratio in the range of 0.9-1.6:1.

10. A multi-stage process for the production of hydrocarbons from syngas comprising hydrogen and carbon monoxide, which comprises the steps of:
a) providing at least three Fischer-Tropsch subreactors, wherein each subreactor is connected on the inlet side to a feed for fresh syngas feed, a feed for an additional hydrogen-containing gas, and a feed for recycle gas and wherein each subreactor is connected on the outlet side to an effluent line, wherein all effluent lines are connected to a separation unit;
b) providing a fresh syngas feed to at least one and less than all Fischer-Tropsch subreactors, and allowing CO and hydrogen to convert in these subreactor(s) into hydrocarbon products in the second stage Fischer-Tropsch reactor at a temperature in the range from 125 to 400° C. and a pressure in the range from 5 to 150 bar absolute, and a gaseous hourly space velocity in the range from 500 to 10000 Nl/l/h, wherein the subreactors provided with fresh syngas feed are not simultaneously provided with additional hydrogen-containing gas;
c) feeding the effluent from the subreactors provided with fresh syngas to a separation unit;
d) removing a gasous effluent stream comprising hydrogen and CO from the separation unit;
e) removing one or more other streams comprising hydrocarbon and/or water from the separation unit;
f) conveying a first portion of the gaseous effluent stream to the at least one and less than all Fischer-Tropsch subreactors which are not provided with fresh syngas, and allowing CO and hydrogen to convert in these subreactor(s) into hydrocarbon products in the second stage Fischer-Tropsch reactor at a temperature in the range from 125 to 400° C. and a pressure in the range from 5 to 150 bar absolute, and a gaseous hourly space velocity in the range from 500 to 10000 Nl/l/h;
g) feeding the effluent from the subreactors provided with a first portion of the gaseous effluent stream to the separation unit; and
h) removing a second portion of the gaseous effluent stream as off-gas.

11. A process according to claim 10 wherein an additional hydrogen-containing gas stream is provided to the at least one and less than all Fischer-Tropsch subreactors which are not provided with fresh syngas.

12. A process according to claim 10, wherein a third portion of the gaseous effluent stream is conveyed to the at least one and less than all Fischer-Tropsch subreactors which are provided with fresh syngas feed.

13. A process according to claim 10, wherein during operation for at least one of the subreactors which are provided with a first portion of the gaseous effluent stream and an additional hydrogen-containing gas stream, the provision of an additional hydrogen-containing gas stream is discontinued, and the provision of a fresh syngas feed is started.
